(19) United States
(12) Patent Application Publication
(10) Pub. No.: US 2017/0245839 A1
(43) Pub. Date: Aug. 31, 2017

(54) SPECIMEN RETRIEVAL SYSTEMS

(71) Applicant: Covidien LP, Mansfield, MA (US)

(72) Inventor: Jaroslaw T. Malkowski, Trumbull, CT (US)

(21) Appl. No.: 15/504,340

(22) PCT Filed: Jul. 22, 2015
(86) PCT No.: PCT/US15/41468

§ 371 Date: Feb. 16, 2017

Related U.S. Application Data
(60) Provisional application No. 62/039,055, filed on Aug. 19, 2014.
(60) Provisional application No. 62/039,048, filed on Aug. 19, 2014.
(60) Provisional application No. 62/039,041, filed on Aug. 19, 2014.

(51) Int. Cl. A61B 10/02; A61B 17/32; A61B 17/34

(52) U.S. Cl. CPC A61B 10/02; A61B 17/3421; A61B 17/32002; A61B 2017/00287

(57) ABSTRACT

A system for morcellating tissue within a body cavity is provided. The system includes a bag guide having a tubular body with proximal and distal ends, the distal end including a flange, a morcellator including an enlarged mouth for receipt within the flange of the bag guide, and a specimen bag receivable between the flange of the bag guide and the enlarged mouth of the morcellator. Also provided is a method of morcellating tissue within a body cavity.

### CROSS-REFERENCE TO RELATED APPLICATIONS

The present application claims the benefit of and priority to U.S. Provisional Patent Application Ser. No. 62/039,041, filed Aug. 19, 2014; U.S. Provisional Patent Application Ser. No. 62/039,048, filed Aug. 19, 2014; and, U.S. Provisional Patent Application Ser. No. 62/039,055, filed Aug. 19, 2014. The contents of each of these applications are incorporated by reference herein in their entirety.

### BACKGROUND

Technical Field

The present disclosure relates generally to tissue removal during minimally invasive surgical procedures using specimen bags. More particularly, the present disclosure relates to methods and/or apparatuses for morcellating tissue within a specimen bag within a body cavity.

Background of Related Art

During a minimally invasive surgical procedure, such as, for example, a laparoscopic cholecystectomy, following placement of the gallbladder or other tissue to be removed within a specimen bag, the open end of the specimen bag is retracted through an incision in the abdominal wall to permit access to the interior of the specimen bag. A morcellator may then be received within the specimen bag to morcellate and, in most instances remove, the tissue. Once the tissue is removed from the specimen bag or once the tissue has been sufficiently reduced in size to permit removal of the specimen bag through the incision, the specimen bag is removed through the incision. Without visualizing the contents of the specimen bag, it is difficult for a surgeon to ensure that the tissue has been sufficiently morcellated and/or removed from within the specimen bag to permit removal of the specimen bag from the body cavity.

It would, therefore, be advantageous to provide specimen bags and methods of using the specimen bags that facilitate sufficient morcellation of the contents of the specimen bags and/or removal of the contents of the specimen bags from within the specimen bags during a laparoscopic procedure within a body cavity.

### SUMMARY

A system for morcellating tissue within a body cavity is provided. The system includes a bag guide having a tubular body with proximal and distal ends, the distal end including a flange, a morcellator including an enlarged mouth for receipt within the flange of the bag guide, and a specimen bag receivable between the flange of the bag guide and the enlarged mouth of the morcellator.

In embodiments, the flange includes a bell shape. The morcellator may be receivable through the tubular body of the bag guide. The flange may include a plurality of fingers. The plurality of fingers may be flexible in a radially inward direction. The plurality of fingers may be fixed in a radially outward direction. The system may further include a trocar receivable through an opening in tissue. The system may also or instead include a containment bag received about the specimen bag and the specimen bag may be porous. The system may further including a source of insufflation gas.

Also provided is a method for morcellating tissue within a body cavity. The method includes positioning a bag guide through an opening in tissue, receiving a specimen bag through the bag guide, placing tissue with the specimen bag through an open end of the specimen bag, retracting an open end of the specimen bag through the bag guide, positioning a morcellator within the specimen bag and through the bag guide such that the specimen bag is disposed between the bag guide and the morcellator, and retracting the specimen bag relative to the morcellator.

In embodiments, positioning a bag guide includes receiving a flange of the bag guide within a body cavity of a patient. Positioning a morcellator may include receiving an enlarged mouth of the morcellator within the flange of the bag guide. Receiving a bag guide through an opening in tissue may include receiving an access port through the opening in tissue and receiving the bag guide through the access port. The method may further include attaching a source of insufflation fluid to the access port.

A system for morcellating tissue within a body cavity is provided. They system includes a specimen bag, a source of cryogenic liquid, and an instrument for impacting tissue within the specimen bag.

Also provided is a method for morcellating tissue within a body cavity. The method includes receiving tissue within a specimen bag freezing the tissue within the specimen bag using a cryogenic liquid, and engaging the frozen tissue with an instrument to shatter the tissue.

Another method for morcellating tissue within a body cavity is provided, the method includes receiving tissue within a specimen bag, freezing a distal end of an instrument using a cryogenic liquid, engaging the tissue within the specimen bag with the frozen distal end of the instrument.

A specimen bag suitable for morcellating tissue therein. The specimen bag including an inner layer formed of a permeable material, and an outer layer formed about the inner layer. An inner surface of the inner layer is coated with a low friction material and an outer surface of the outer layer is coated with a water tight material.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective side view of an access port and a bag guide according to an embodiment of a system for morcellating tissue of the present disclosure;

FIG. 2 is a perspective side view of a specimen bag according to an embodiment of the present disclosure received through the access port and the bag guide shown in FIG. 1;

FIG. 3 is a perspective side view of the access port, the bag guide, and the specimen bag shown in FIG. 2 and further including a morcellator according to an embodiment of the present disclosure;

FIG. 4 is a perspective side view of a specimen bag according to another embodiment of the present disclosure;

FIG. 4A is a cross-sectional view taken along line 4A-4A shown in FIG. 4; and

FIG. 5 is a perspective side view of another embodiment of a system for morcellating tissue of the present disclosure.

### DETAILED DESCRIPTION

Embodiments of the present disclosure will now be described in detail with reference to the drawings, in which like reference numerals designate identical or corresponding elements in each of the several views. As used herein, the term distal refers to the portion of the instrument which is farthest from the user, while the term proximal refers to that portion of the instrument which is closest to the user. In the following description, well-known functions or constructions are not described in detail to avoid obscuring the present disclosure in unnecessary detail.

As used herein with reference to the present disclosure, the terms laparoscopic and endoscopic are interchangeable and refer to instruments having a relatively narrow operating portion for insertion into a cannula or a small incision in the skin. Laparoscopic and endoscopic also refer to minimally invasive surgical procedures. It is believed that the present disclosure may find use in any procedure where access to the interior of the body is limited to one or more relatively small incisions, with or without the use of a cannula or other access port, as in minimally invasive procedures.

Various specimen bags, instruments, and methods for inserting and retrieving the specimen bags from within a patient are known. For example, commonly owned U.S. Pat. Nos. 5,647,372, 5,465,731 6,409,733 5,037,379, and 5,735,289, and U.S. Patent Application Publication No. 2014/0135788 disclose various specimen bags, applicators, and methods for deploying the specimen bags. The contents of these patents and publications are incorporated by reference herein in their entirety.

The aspects of the present disclosure may be modified for use with various methods for retrieving tissue during minimally invasive procedures. Although the embodiments of the present disclosure rill be described with reference to a cholecystectomy, e.g., gallbladder removal, the embodiments of the present disclosure may be used or modified for use with other minimally invasive procedures, e.g., appendectomies, nephrectomies, colectomy, splenectomy. Unless otherwise noted, the specimen bags of the present disclosure are formed of rip stop nylon or other suitable material. The specimen bags of the present disclosure may be closed using a drawstring or in any other suitable manner, and may include any feature necessary for deploying and/or retrieving the specimen bag from within a body cavity.

During laparoscopic procedures, damaged tissue is separated from surrounded tissue to permit removal of the tissue from a body cavity. In many instances, it is not possible to remove the damaged tissue through the small incisions used in laparoscopic procedures, thereby necessitating morcellation of the tissue. Embodiments of the present disclosure relate to systems and methods for morcellating tissue within a body cavity during a laparoscopic procedure.

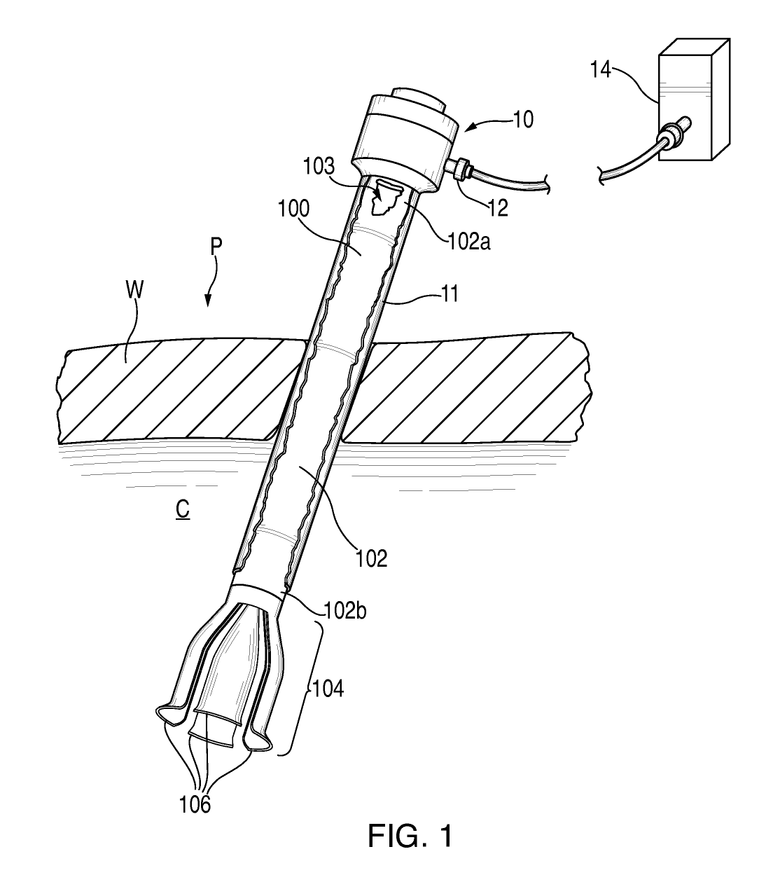

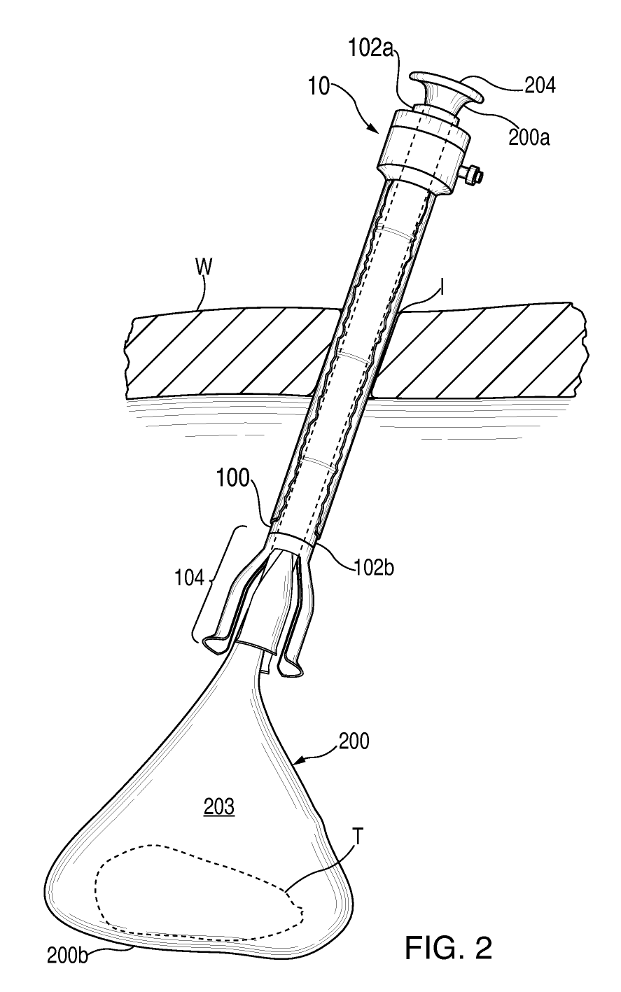

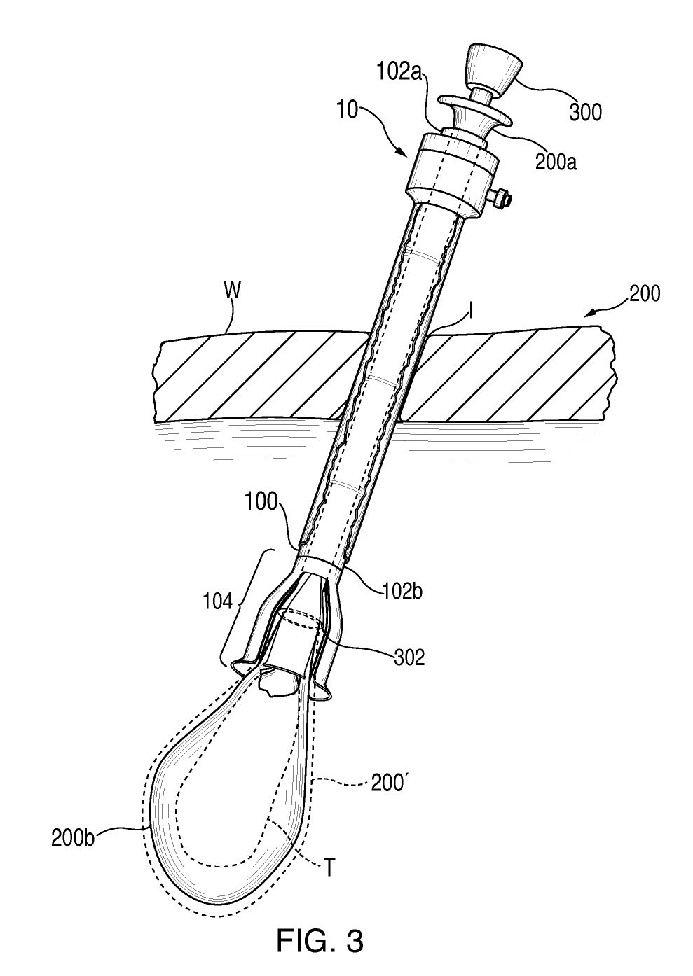

With reference to FIGS. 1-3, a system for morcellating tissue is shown. The system includes an access port 10, a bag guide 100, a specimen bag 200 (FIG. 2), and a morcellator 300 (FIG. 3). Referring initially to FIG. 1, the access port 10 includes a conventional trocar or other device for providing access to a body cavity “C” through an incision “I” in an abdominal wall “W” of a patient “P”. The access port 10 defines a central lumen 11 for providing access to the body cavity “C”. The access port 10 may include an insufflation valve 12 for communicating with a source of insufflation gas 14 (FIG. 1) for providing insufflation gas to the body cavity “C” and/or to the specimen bag 200 (FIG. 2).

With reference still to FIG. 1, the bag guide 100 includes an elongated tube 102 having a proximal end 102a and a distal end 102b and defining a longitudinal passage 103 extending therethrough. The bag guide 100 may be formed of thin wall stainless steel or any other durable material. A flange 104 is formed on the distal end 102b of the elongate tube 102. The flange 104 defines a substantial bell shape and includes a plurality of fingers 106. The fingers 106 are configured to flex radially inward to permit insertion of the flange 104 through the central lumen 11 of the access port 10. The fingers 106 are also fixed from flexing in a radially outward direction. By resisting outward radial flexion, as the specimen bag 200 is retracted through the flange 104 of the bag guide 100, the fingers 106 of the flange 104 confine and direct the specimen bag 200 within the flange 104 of the elongate tube 102 and into the longitudinal passageway 103.

With reference now to FIG. 2, the specimen bag 200 includes an open end 200a and a closed end 200b and defines a cavity 203. In embodiments, the specimen bag 200 is formed of rip stop nylon or other suitable material. The material forming the specimen bag 200 is of sufficient strength to permit retraction of the specimen bag 200 through the bag guide 100 without tearing. The open end 200a of the specimen bag 200 may include a lip 204 or other feature for facilitating grasping and retraction of the specimen bag 200, as will be described in further detail below.

Turning to FIG. 3, the morcellator 300 includes any surgical instrument for cutting, mincing up, liquefying, or morcellating, tissue into smaller pieces. The morcellator 300 may be powered or hand-operated, and may be configured to extract the tissue from the specimen bag, via, e.g., a vacuum tube or through the operation of a cutting mechanism (not shown), as the tissue is morcellated. The morcellator 300 includes an enlarged mouth 302 configured to be received through the trocar 10 and within the flange 104 on the distal end 102b of the elongate tube 102 of the bag guide 100. The enlarged mouth 302 of the morcellator 300 is dimensioned to protect the specimen bag 100 from contact with the cutting mechanism of the morcellator 300. The enlarged mouth 302 of the morcellator is further dimension to permit receipt and retraction of the specimen bag 200 between the enlarged mouth 302 of the morcellator 300 and the flange 104 of the guide bag 100.

The operation of system according to the present disclosure will now be described with reference to FIGS. 1-3. Initially, the body cavity “C” of the patient “P” is accessed using traditional laparoscopic methods and the tissue to be removed from the body, e.g., gallbladder “G”, is separated from the surrounding tissue. The accessing of the body cavity “C” and the separation of the gallbladder “G” from the surrounding tissue may be performed through the trocar 10 or by other means. Referring now to FIG. 1, the trocar 10 or other suitable access device is received through the incision “I”. The bag guide 100 is then received through the central lumen 11 of the trocar 10. The fingers 106 of the flange 104 of the bag guide 100 are flexed radially inward to facilitate receipt of the bag guide 100 through the trocar 10. The bag guide 100 is advance through the trocar 10 until the flange 104 on the distal end 102b of the elongate tube 102 of the bag guide 100 is received with the body cavity “C”.

The specimen bag 200, if not already disposed within the body cavity “C”, may be received through the longitudinal passageway 103 of the bag guide 100 or using other means. After receiving the specimen bag 200 within the body cavity “C”, the gallbladder “G” is positioned within the specimen bag 200. The open end 200a of the specimen bag 200 is then received through the bag guide 100 such that the open end 200a of the specimen bag 200 extends from the proximal end 102a of the elongate tube 102 of the bag guide 100. Graspers (not shown) or another instrument may be used to direct the open end 200a of the specimen bag 200 through the bag guide 100. Alternatively, the specimen bag 200 may include a draw string (not shown) or other feature for assisting in retraction of the open end 200a of the specimen bag 200 through the bag guide 100. As noted above, the open end 200a of the specimen bag 200 may include a lip 204 for facilitating receipt of the open end 200a of the specimen bag 200 through the bag guide 100.

Turning to FIG. 3, the morcellator 300 is then received through the open end 200a of the specimen bag 200 and within the longitudinal passageway 103 of the bag guide 100 until the enlarged mouth 302 of the morcellator 300 is disposed within the flange 104 of the bag guide 100. In this manner, the enlarged mouth 302 of the morcellator 300 is received within the cavity 203 of the specimen bag 200 and the specimen bag 200 is received between the enlarged mouth 302 of the morcellator 300 and the flange 104 of the bag guide 100. The morcellator 300 is then activated.

Subsequent to activation of the morcellator 300, the open end 200a of the specimen bag 200 is retracted through the trocar 10 and the bag guide 100 relative to the morcellator 300. As the specimen bag 200 is retracted through the bag guide 100, the fingers 106 of the flange 104 of the bag guide 100 confine the specimen bag 200 and direct the contents of the specimen bag 200 towards the enlarged mouth 302 of the morcellator 300. Continued retraction of the specimen bag 200 through the bag guide 100 ensures that the contents of the specimen bag 200 are brought into contact with the enlarged mouth 302 of the morcellator 300. In embodiments, the open end 200a of the specimen bag 200 is rolled as the specimen bag 200 is retracted through the trocar 10.

The specimen bag 200 is refracted through the bag guide 100 until the closed end 200b of the specimen bag 200 engages the enlarged mouth 302 of the morcellator 300 to ensure that the contents of the cavity 203 of the specimen bag 200 are morcellated. The morcellator 300 is then removed from within the bag guide 100 and the specimen bag 200 is retracted through the bag guide 100. Alternatively, the bag guide 100 and the specimen bag 200 are retracted through trocar 10 together.

In an alternative embodiment, as the specimen bag 200 is formed of a porous material and is received within a second, fluid tight bag 200′ (FIG. 3). As the specimen bag 200 is retracted relative to the morcellator 300, the morcellated tissue and resulting fluids pass through the porous specimen bag 200 and into the second bag 200′. Upon complete refraction of the specimen bag 200 relative to the morcellator, i.e., once the contents of the cavity 203 of the specimen bag 200 are completely morcellated, the specimen bag 200 and the second bag 200′ are removed from the body cavity “C” as described above.

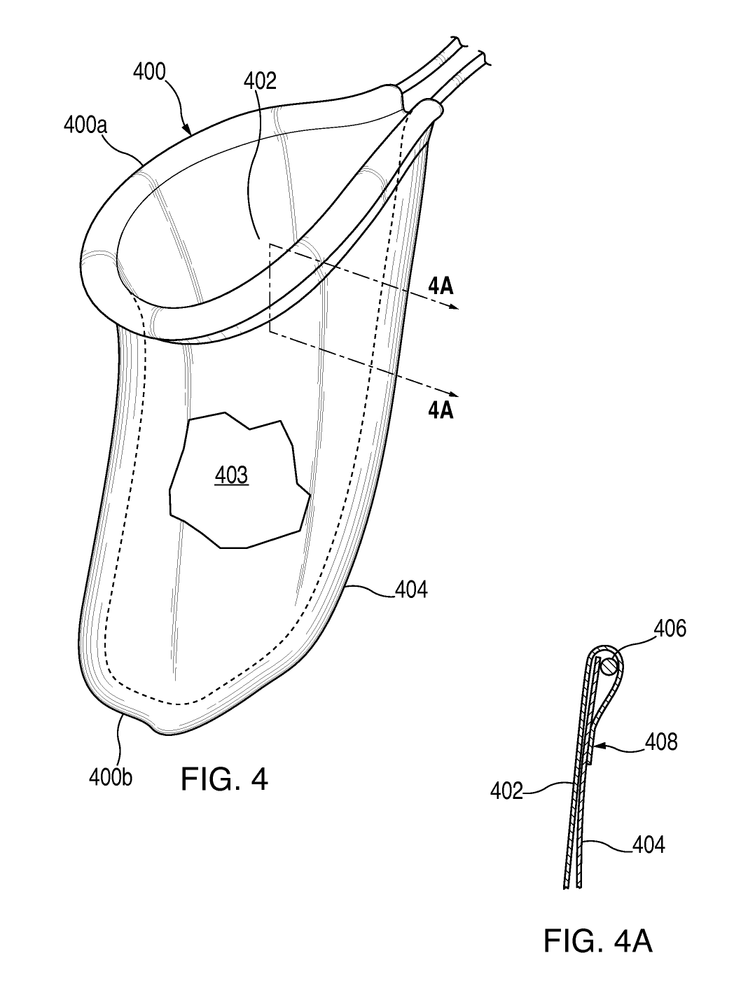

With reference now to FIGS. 4 and 4A, a specimen bag according to an embodiment of the present disclosure is shown generally as specimen bag 400. The specimen bag 400 includes an open end 400a and a closed end 400b and defines a cavity 403. The specimen bag 400 includes an inner layer 402 and an outer layer 404. The inner layer 402 is formed of a cut resistant polymer, e.g., Vectran LCP fiber, para-aramid, ultra-high-molecular-weight polyethylene (UHMWP), aromatic polyester. The inner layer 402 may include a plurality of metal strands/fibers, e.g., stainless steel, tungsten. The outer layer 404 is formed of a fluid proof film or coated fabric, e.g., urethane, silicone, polyethylene.

In embodiments, the inner layer 402 includes a low friction layer (not shown), e.g., PTFE, Teflon, silicone, on an inner surface, and/or hydrophobic layer. The low friction layer facilitates positioning of tissue within the cavity 403 of the specimen bag 400. Alternatively, the inner layer 402 is formed from a relatively low friction material.

The specimen bag 400 may be closed using a drawstring 406 or in any other suitable manner and may include any feature necessary for deploying and/or retrieving the specimen bag 400. As shown, in FIG. 4A, the inner layer 402 of the specimen bag 400 is secured to the outer layer 404 along a seam 408 formed in the open end 400a of the specimen bag 400. Alternatively, the seam 408 may extend about a part or all of the outer periphery of the specimen bag 400. The seam 408 may be formed by stitching, bonding, or in any other suitable manner. By securing inner layer 402 to the outer layer 404 along the seam 408, the remainder of the inner layer 402 and the outer layer 404 may move independently of one other, thereby increasing the pliability of the specimen bag 400. The independent construction of the inner and outer layers 402, 404 of the specimen bag 400 may also permit fluids to be evacuated from the inner layer 402 into the outer layer 404 to facilitate removal of the specimen bag 400 from the patient. Alternatively, the inner layer 402 may be partially or entirely secured to the outer layer 404.

In another embodiment of the present disclosure, a cryogenic liquid, e.g., liquid nitrogen or liquid helium, is used to freeze the tissue within the specimen bag. The frozen tissue is then broken into smaller pieces or shattered using forceps or other instrument. Alternatively, a cryogenic liquid is used to freeze a working end of an instrument, e.g., the grasping end of forceps, and engagement of the tissue with the instrument freezes the tissue and allows the tissue to be broken up or shattered.

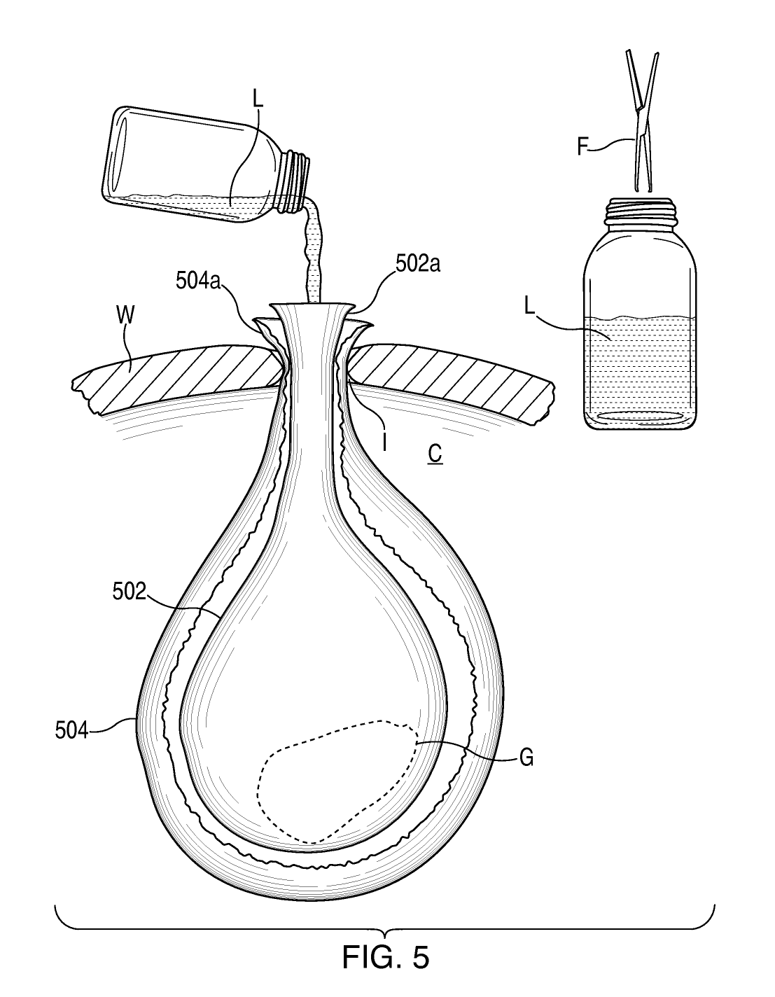

With reference to FIG. 5, in a method of using cryogenic liquid “L” to morcellate the tissue, e.g., gallbladder “G”, the gallbladder “G” is received within a cavity 503 of an inner bag 502 of a specimen bag 500 using know techniques. The inner bag 502 is received within an outer bag 504 to separate the inner bag 502 from surrounding tissue within the body cavity “C” to prevent freezing of the surround tissue when the cryogenic liquid “L” is introduced into the cavity 503 of the inner bag 502 of the specimen bag 500. The open ends 502a and 504a of the inner and outer bags 502, 504, respectively, are then received through an incision “I” in the abdominal wall “W” of the patient. The inner and outer bags 502, 504 are then inflated using known techniques.

The cryogenic liquid “L” is then introduced into the cavity 503 of the inner bag 502 of the specimen bag 500 to freeze the gallbladder “G”. As noted above, the outer bag 504 separates the inner bag 502 from the surrounding tissue within the body cavity “C” to prevent damage to the surrounding tissue. Forceps “F” or other instrument is then introduced into the cavity 503 of the inner bag 502 of the specimen bag 500 and engaged with the frozen gallbladder “G” to cause the gallbladder “G” to shatter or otherwise be broken into smaller pieces. The specimen bag 500 and the morcellated tissue may then be removed from the body cavity “C” through the incision “I”.

As noted above, in an alternative method of morcellating tissue using cryogenic liquid “L”, a distal end of the forceps “F” are frozen using the cryogenic liquid “L”. The frozen distal end of the forceps “F” are then introduced into the cavity 503 of the inner bag 502 of the specimen bag 500 and engaged with the gallbladder “G” to cause the freezing of gallbladder “G”. Engagement of the gallbladder “G” with the frozen distal end of the forceps “F” cause the gallbladder “G” to be broken into smaller pieces to facilitate removal of the specimen bag 500 from within the body cavity “C” through the incision “I”.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

1. A system for morcellating tissue, the system comprising:
a bag guide including a tubular body having a proximal end and a distal end, the distal end including a flange;
a morcellator including an enlarged mouth for receipt within the flange of the bag guide; and
a specimen bag receivable between the flange of the bag guide and the enlarged mouth of the morcellator.

2. The system of claim 1, wherein the flange includes a bell shape.

3. The system of claim 1, wherein the morcellator is receivable through the tubular body of the bag guide.

4. The system of claim 1, wherein the flange includes a plurality of fingers.

5. The system of claim 4, wherein the plurality of fingers are flexible in a radially inward direction.

6. The system of claim 5, wherein the plurality of fingers are fixed in a radially outward direction.

7. The system of claim 1, further including a trocar receivable through an opening in tissue.

8. The system of claim 1, further including a containment bag received about the specimen bag.

9. The system of claim 8, wherein the specimen bag is porous.

10. The system of claim 7, further including a source of insufflation gas.

11. A method for morcellating tissue, the method comprising:
positioning a bag guide through an incision in tissue;
receiving a specimen bag through the bag guide;
placing tissue with the specimen bag through an open end of the specimen bag;
retracting an open end of the specimen bag through the bag guide;
positioning a morcellator within the specimen bag and through the bag guide such that the specimen bag is disposed between the bag guide and the morcellator; and
retracting the specimen bag relative to the morcellator.

12. The method of claim 11, wherein positioning a bag guide includes receiving a flange of the bag guide within a body cavity of a patient.

13. The method of claim 12, wherein positioning a morcellator includes receiving an enlarged mouth of the morcellator within the flange of the bag guide.

14. The method of claim 11, wherein receiving a bag guide through an opening in tissue includes receiving an access port through the incision in tissue and receiving the bag guide through the access port.

15. The method of claim 14, further including attaching a source of insufflation fluid to the access port.
